(19) United States
(12) Patent Application Publication
(10) Pub. No.: US 2008/0077205 A1
(43) Pub. Date: Mar. 27, 2008

(54) Cooling-normothermic-heating device with activated negative pressure system

(75) Inventor: Karl Hans Cazzini, Orchard Park, NY (US)

Correspondence Address: Kevin D. McCarthy;Roach Brown McCarthy & Gruber, P.C., 1620 Liberty Building, 420 Main Street, Buffalo, NY, 14202, US

(73) Assignee: Gaymar Industries, Inc.

(21) Appl. No.: 11/528,152

(22) Filed: Sep. 27, 2006

(51) Int. Cl. A61F 7/00

(52) U.S. Cl. 607/104; 607/108

(57) ABSTRACT

The present invention is directed to using a thermoregulatory sensor in conjunction with a cooling-normothermic-heating device that applies a desired thermal energy to a target heat exchange surface of a mammal that is under negative pressure. The thermoregulatory sensor, unlike the prior art, does not initiate and/or control the thermal energy applied to the mammal. Instead the thermoregulatory sensor initiates, controls and/or manages the negative pressure applied to the target heat exchange surface. By initiating, controlling and/or managing the negative pressure, (1) the vasodilation of the target heat exchange surface is also controlled and/or managed and/or (2) thermal communion between the exchange surfaces (skin and heat exchanger) is controlled, and/or managed.

### FIELD OF THE INVENTION

The field of this invention relates generally to thermoregulatory status of mammals, and more particularly to the control and management of vasodilation within portions of mammalian bodies.

### BACKGROUND OF THE INVENTION

Mammalian body temperature is normally controlled by an internal autonomic regulatory system referred to in this specification as the thermoregulatory system. Controlling blood flow to specialized skin areas of the body at non-hairy skin surfaces (i.e., at the palms, soles of the feet, cheeks/nose regions) is an important aspect of the thermoregulatory system. Subcutaneous to these areas, there are unique anatomical vascular structures called venous plexuses. These structures serve to deliver large volumes of blood adjacent the skin surface. By this delivery of blood, significant heat transfer is enabled for the maintenance of internal organs within a functional temperature range. Blood is permitted to pass through the venous plexuses “radiator” structures by way of arterio venous anastomoses, or AVAs that gate or control the arterial input side of the venous plexuses. Thus, the AVA's serve an integral part of the heat transfer system, providing thermoregulatory control. Together, the AVA's and venous plexuses comprise a body's relevant heat exchange vasculature.

Normally, when body and/or environmental temperatures are high, dilation of certain blood vessels favors high blood flow to the noted heat exchange surfaces, thus increasing heat loss to the environment and reduction in the deep body core region temperature. As environmental and/or body temperatures fall, vasoconstriction reduces blood flow to these surfaces and minimizes heat loss to the environment.

There are situations, however, in which it would be desirable to manipulate the transfer of heat across skin surfaces to lower and/or raise the core body temperature. Such core body cooling or heating would be useful in a number of applications, including surgical situations, therapeutic treatment regimens and as a component of improving athletic or industrial performance.

The present invention is geared to improve the implementation of these goals. It does so in various ways by specifically taking natural vasoconstriction tendencies into account in order that unintended vasoconstriction (during an intended procedure) will not adversely effect blood flow in the region of a heat transfer surface so as to prevent adequate heat transfer.

In U.S. Pat. No. 5,683,438, Grahn et al. disclosed a heating device that encloses a patient's body part under negative pressure and applies a warm temperature to the body part. The warm temperature could be provided by a heat lamp, a thermal blanket, or a metallic tube having a warm liquid medium passing and/or contained in the tube. Grahn discloses in U.S. Pat. No. 6,656,208 that the '438 patent only discloses a hard seal embodiment for enclosing the patient's body part. In the '208 patent, Grahn discloses a hard seal “is characterized as one designed to altogether avoid air leakage past the boundary it provides. In theory, a “hard” seal will allow a single evacuation of the negative pressure chamber for use in the methods. In practice, however, a “hard” seal can produce a tourniquet effect. Also, any inability to maintain a complete seal will be problematic in a system requiring as much.” Recognizing a problem with the '438 patent, Grahn submitted the application that matured into the '208 patent.

The '208 patent discloses the same device as disclosed in the '438 patent except it (a) applies “a cool temperature to the patient's body at a temperature that avoids local vasoconstriction” and (b) uses, allegedly, a soft seal to enclose the patient's body part. A soft seal “is characterized as providing an approximate or imperfect seal at a user/seal interface. Such a seal may be more compliant in its interface with a user. Indeed, in response to user movement, such a seal may leak or pass some air at the user/seal interface. In a negative-pressure system designed for use with a soft seal, a regulator or another feedback mechanism/routine will cause a vacuum pump, generator, fan or any such other mechanism capable of drawing a vacuum to respond and evacuate such air as necessary to stabilize the pressure within the chamber, returning it to the desired level. Active control of vacuum pressure in real-time or at predetermined intervals in conjunction with a “soft” seal provides a significant advantage over a “hard” seal system that relies on simply pulling a vacuum with the hopes of maintaining the same.” To one of ordinary skill in the art, Grahn disclosed a system that (1) measures the negative pressure within the enclosure, (2) transmits a signal to a negative pressure generator, and (3) in response to the signal received, the negative pressure generator stabilizes the negative pressure within the enclosure. This method will be referred to as the “Stabilizer Protocol.”

It should be noted that Grahn et al. admitted that its hard seal leaks in the operation manual of their first commercial embodiment of the device called Thermo-STAT. The Thermo-STAT device operation manual was publicly available prior to Apr. 20, 1999.

In published patent application 2005-0103353, Grahn et al. disclose, “Negative pressure includes conditions where a pressure lower than ambient pressure under the particular conditions in which the method is applied, e.g., 1 ATM at sea level. The magnitude of the decrease in pressure from the ambient pressure under the negative pressure conditions in one example is at least about 20 mmHg, preferably at least 30 mmHg, and more preferably at least about 35 mmHg, where the magnitude of the decrease may be as great as 85 mmHg or greater, but preferably does not exceed about 60 mmHg, and more preferably does not exceed about 50 mmHg. When the method is performed at or about sea level, the pressure under the negative pressure conditions generally may range from about 740 to 675 mmHg, preferably from about 730 to 700 mmHg and more preferably from about 725 to 710 mmHg.

In practicing the exemplary methods, the negative pressure conditions during contact with the skin of a subject may be static/constant or variable. Thus, in certain examples, the negative pressure is maintained at a constant value during contact of the surface with the low temperature medium. In yet other examples, the negative pressure value is varied during contact, e.g., oscillated. Where the negative pressure is varied or oscillated, the magnitude of the pressure change during a given period may be varied and may range from about 85 to 40 mmHg, and preferably from about 40 to 0 mmHg, with the periodicity of the oscillation ranging from about 0.25 sec to 10 min, and preferably from about 1 sec to 10 sec.

Further discussion of suitable vacuum/negative pressure approaches are described in the U.S. Pat. No. 6,602,277 noted above as well as U.S. Pat. No. 5,683,438 to Grahn and . . . [U.S. Pat. No. 6,656,208; and U.S. Pat. No. 6,673,099] to Grahn, et al.—all of which are incorporated herein by reference in their entireties. Any other details informing the operation of the present invention may be drawn from one or more of these four sources, or be provided by application of the talents of one with ordinary skill in the art.”

In the '438 patent, Grahn et al. disclosed, “the predetermined negative pressure is oscillated for promoting the transport of the thermal energy to the core body of the mammal by its own circulatory system . . . . To further aid the body in absorbing the thermal energy delivered, the negative pressure value can be changed. For example, a periodic fluctuation or oscillation between −20 mmHg and −85 mmHg may be introduced. The period can be in rhythm with the patient's heart rate. This oscillation will maximize the heat transfer to the core body.” This method will be referred to as the “Heart Rate Protocol” for controlling the negative pressure within the enclosure.

Other than the Heart Rate Protocol and the Stabilizer Protocol, we were unable to find in any Grahn et al. reference or references cited in a Grahn et al. reference any other reason concerning when to alter (and/or oscillate) the negative pressure in the enclosure. As for turning the negative pressure on, the prior art discloses that the negative pressure is initiated only when the thermal energy unit is activated.

In many of Grahn's published patent applications and issued U.S. patents, Grahn et al. disclose heating or cooling devices capable of detecting a need for thermal energy input with a target heat exchange surface for a requisite period of time. Many of those devices have a sensing element for detecting a requirement for thermal energy input—in most cases, vasoconstriction and/or vasodilation. Those detection and measurement values correspond with initiating and/or altering the thermal energy applied to the patient, not initiating and/or altering the negative pressure applied. At best, the negative pressure is altered only when the negative pressure in the enclosure is not in a preselected parameter of negative pressure in the enclosure.

### SUMMARY OF THE INVENTION

The present invention is directed to using a thermoregulatory sensor in conjunction with a cooling-normothermic-heating device that applies a desired thermal energy to a target heat exchange surface of a mammal that is under negative pressure. The thermoregulatory sensor, unlike the prior art, does not initiate and/or control the thermal energy applied to the mammal. Instead the thermoregulatory sensor initiates, controls and/or manages the negative pressure applied to the target heat exchange surface. By initiating, controlling and/or managing the negative pressure, (1) the vasodilation of the target heat exchange surface is also controlled and/or managed and/or (2) thermal communion between the exchange surfaces (skin and heat exchanger) is controlled, and/or managed.

### BRIEF DESCRIPTION OF THE FIGURES

Each of the figures diagrammatically illustrates aspects of the invention. Of these figures:

FIG. 1 illustrates a front view of a cooling-normothermic-heating transfer device for mammalian bodies;

FIG. 2 illustrates a rear view of FIG. 1;

FIG. 3 illustrates a cross-sectional view of FIG. 2 taken along lines 3-3; and

FIG. 4 illustrates an alternative embodiment of the invention.

### DETAILED DESCRIPTION

The present invention is a cooling-normothermic-heating device 10. The device 10 has a housing 12 defining a negative pressure chamber 14, a heat-exchange element 16 and a seal 18.

### Housing

Housing 12 may be made from a cover 22 and a base 24. The housing 12 could be of numerous shapes designed to enclose a portion of a patient's body. The portion that contacts the heat exchange element 16 is referred to as a target heat exchange surface. The portion of the patient's body can be a foot, leg, feet, legs, arm(s), hand(s) or combinations thereof.

Housing 12 may be constructed from multiple pieces, including an end cap 26 as shown, or it may be provided as a unitary structure. Cap 26 is shown having ports 28. A first port may be utilized for connection to a vacuum source, while the second may be utilized for a vacuum gauge. Of course, alternate port placement is also possible.

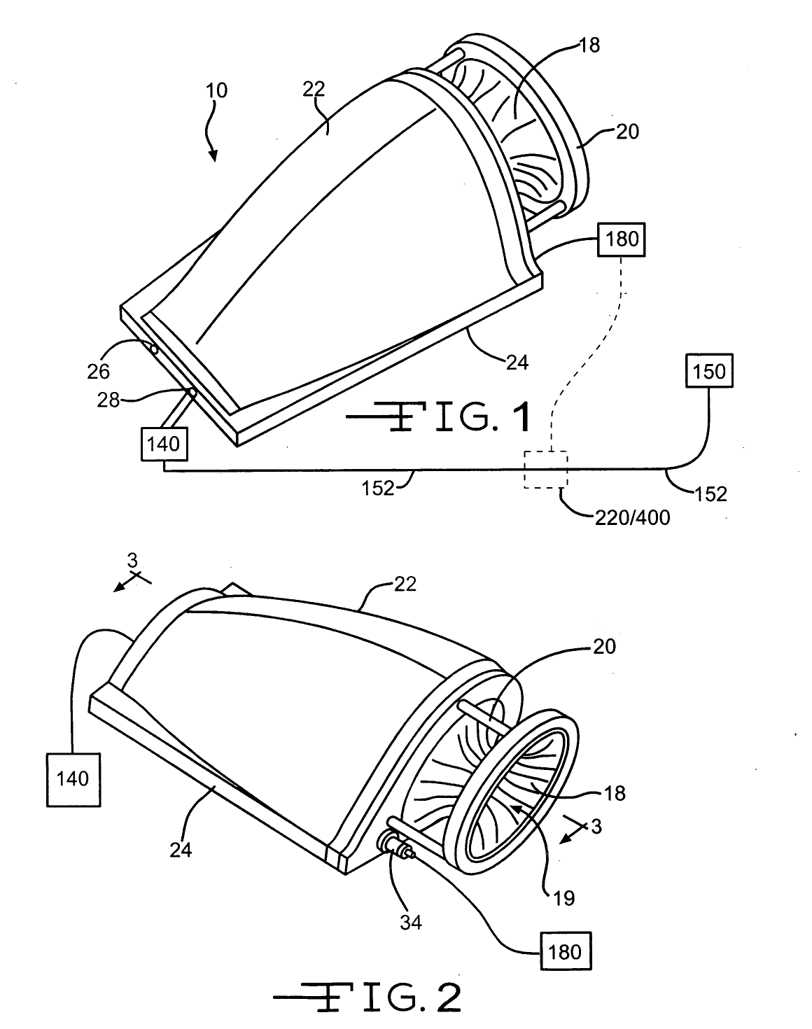

Negative pressure chamber 14 is preferably provided between heat exchange element 16 and cover 22 as shown in FIG. 1, or surrounded by housing 12 with the heat exchange element 16 in and/or near the middle of the housing as shown in FIG. 4. The negative pressure generated in the negative pressure chamber 14 is the result from any device 140 (vacuum like generator) that can create the desired negative pressure in the chamber 14. The device 140 can be independent from the housing 12 as illustrated in FIG. 1 or a part of the housing 12 as illustrated in FIG. 4. In any case, the device 140 is electrically interconnected with a biofeedback sensor 150 when the device 10 is being used with a patient. The biofeedback sensor 150 contacts at least a portion of the patient when the device is used in association with the patient.

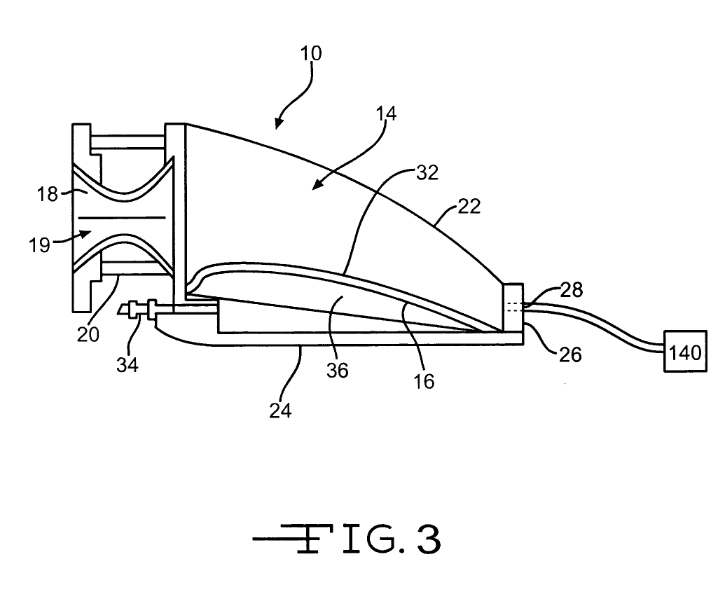

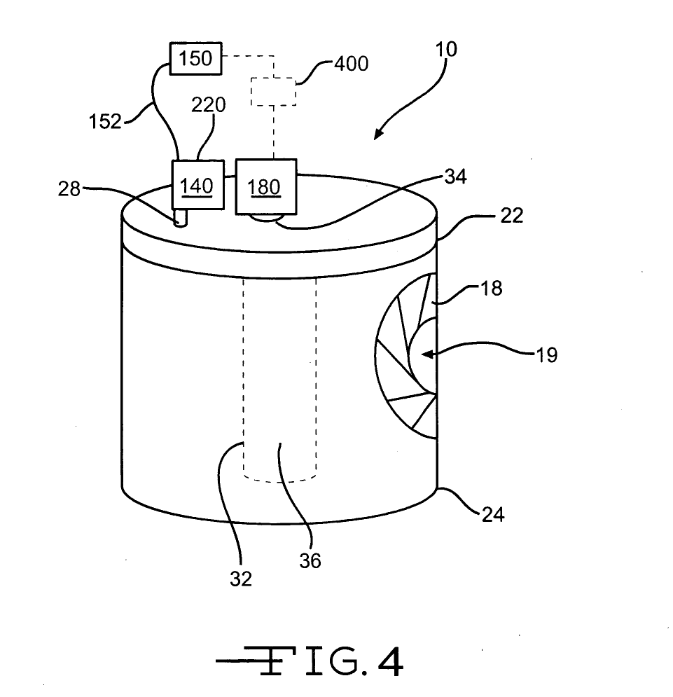

Heat exchange element 16 is preferably made of a thermally conductive material, like and not limited to aluminum. It may be in communication with a Peltier device, a desiccant cooling device, an endothermic chemical reaction, or an exothermic chemical reaction to provide a desired temperature to the target surface area. These chemicals and/or devices can be positioned in a cavity 36 between the element 16 and the base 24 as illustrated in FIG. 1 (or within the element 16 for the embodiment illustrated in FIG. 4), and be inserted into and/or electrically connected through an inlet/outlet(s) 34 as illustrated in FIGS. 2, 3, and 4. More preferably heat exchange member 16 is in communication with at the inlet and the outlet 34 to accommodate a flow of perfusion fluid (liquid and/or gas) behind (as illustrated in FIGS. 1-3) the heat exchange surface 32 or within (as illustrated in FIG. 4) element 16. Chilled or heated water may be used to maintain the contact surface of the element at a desired temperature. Optimally, perfusion fluid is run through a series of switchbacks in the cavity 36, or within the element 16.

The device 10 uses a conventional soft or hard seal 18 to enclose at least the target heat exchange surface in the negative pressure chamber 14. The seal 18 could be polymeric material, webbing as illustrated in FIGS. 1, 2 and 3 with seal supports 20, or a conventional mechanical iris-like design that opens and closes as illustrated in FIG. 4. In any seal embodiment, the seal 18 must have an aperture 19 to allow the patient's body part enter into the negative pressure chamber 14.

The device 10 also has the biofeedback sensor 150 that measures and/or detects a biofeedback parameter and transmits a sensor signal 152 regarding the measurement and/or detected biofeedback parameter directly to or indirectly to the negative pressure generator 140 that provides, controls and/or manages the negative pressure within the negative pressure chamber 14 in response to the sensor's signal.

### Sensor 150

Various methods and devices may be used for determining a characteristic associated with vasoconstriction or vasodilation in a body portion. In one exemplary method for determining whether a body portion is in a vasoconstriction or vasodilation state, the body portion is monitored by measuring blood flow in the particular body portion. Normally, when body and/or environmental temperatures are high, the dilation of certain blood vessels favors high blood flow to these surfaces, and as environmental and/or body temperatures fall, vasoconstriction reduces blood flow to these surfaces and minimizes heat loss to the environment. As such, measuring the blood flow rate in a body portion provides a measure of whether the body portion is in a state of vasoconstriction or vasodilation.

In another exemplary method for measuring vasoconstriction or vasodilation, blood flow in the body portion is measured and monitored by laser Doppler blood flowmetry. Laser Doppler measurement of the blood flow in a body portion provides a measure of whether the body portion is in a state of vasoconstriction or vasodilation, since changes in blood flow rate are measured. In one example, a laser Doppler imager integrated into a heat exchange device and directed toward the palm, a finger, or other body portion is used to measure changes in blood flow rate through the body portion.

Alternatively, vasoconstriction or vasodilation may be monitored by measuring the volume of a body portion. It is commonly understood that vasodilation coincides with a greater body portion volume than observed during vasoconstriction owing to increased blood volume within the body portion during vasodilation. As such, a physical change in the volume of a body portion can be correlated to a condition of vasodilation or vasoconstriction. One example of measuring the volume of a body portion would be to immerse the body portion in a fluid medium. Any changes in the body portion volume would be registered by a change in the volume of fluid medium displaced by the body portion. Or, it may be measured by an impedance-type sensor.

Alternatively, vasoconstriction or vasodilation may be monitored by measuring the heat transfer of a body portion. For example, the heat transfer of a body portion is tested by measuring the presence or absence of a temperature gradient when measuring the temperature difference, e.g., between a finger and the corresponding forearm of an arm. The absence of a temperature gradient (indicative of heat transfer to the finger) correlates with a condition of vasodilation in the finger, while a higher temperature in the forearm than in the finger (indicative of no heat transfer to the finger) correlates with a condition of vasoconstriction.

Alternatively, vasoconstriction or vasodilation may be monitored by measuring the heat flux at the skin surface. For example, the heat flux at the skin surface is tested by placing a temperature sensing device between the skin surface and a cooling object in contact with the skin's surface. The temperature at this sensing device will indicate vasoconstriction or vasodilation. A temperature higher than that of the cooling object will indicate vasodilation while a temperature close to that of the cooling object will indicate vasoconstriction because the skin surface will be cooler.

Alternatively, vasoconstriction or vasodilation is monitored by measuring light absorption of a portion of the body. For example, light absorption can be detected using the technique of plethysmography or through use of an infrared pulse oximeter.

Alternatively, vasoconstriction or vasodilation may be monitored by measuring the temperature of the body of a mammal. Any convenient temperature sensing means may be employed, where suitable means include but are not limited to: thermometers, thermocouples, thermoresistors, microwave temperature sensors, and the like. The position and nature of the temperature sensing devices generally depends on the body portion being tested.

Temperature measurement may involve monitoring the core body temperature of a mammal. By core body is meant the internal body region or portion of the mammal, as opposed to the surface of the mammal. Specific core body regions of interest are the core body region of the head, e.g., the deep brain region, and the core body region of the trunk of the mammal, e.g., the thoracic/abdominal region of the mammal. For detecting the core body region temperature of the head, sensor locations of interest include: the auditory canal (tympanic), the oral cavity, and in the case of microwave detection, anywhere on the surface of the head to measure underlying temperature. For detecting thoracic/abdominal core body temperature, sensor locations include: the esophagus, the rectum, the bladder, the vagina, and in the case of microwave detection, anywhere on the surface of the body to measure the underlying temperature.

Alternatively, vasoconstriction or vasodilation may be monitored by measuring the skin temperature of a mammal. For detecting the skin temperature of a mammal, the simple empirical nursing methodology of holding the hand to test for warmth or coldness can be used. In practicing this method of skin temperature measurement, a warm hand is generally associated with vasodilation, while a cold hand is associated with vasoconstriction. The temperature of the skin can also be detected using sensors such as thermocouples, thermometers, thermoresistors, microwave temperature sensors, temperature sensitive liquid crystals, and other temperature measuring devices. Placement of temperature sensors on the skin surface could be at the site of heat transfer or other locations, or a combination of locations. In one example, vasoconstriction or vasodilation may be monitored by measuring changes in skin surface temperature or heat flow from the body across local skin surface area overlying heat exchange vascular structures.

As for these means of monitoring vasoconstriction or vasodilation through temperature observation, note—that only detecting temperature at the location of heat transfer provides a direct measure of local vasoconstriction. However the monitoring is effected (even—for example—by a combination of any two or more of the above approaches), by controlling vasoconstriction or vasodilation in a body portion of a mammal, the vasoconstriction temperature and the heat transfer temperature can be lowered to increase the temperature gradient between the area of the body containing heat exchange vasculature and the environment, thus increasing heat transfer and facilitating core body cooling.

The sensor generates a sensor signal 152 in response to the measurement of the patient. The sensor signal 152 is transmitted to a comparator 220 or equivalent device. The comparator 220 can be positioned in the device 10 as illustrated in FIG. 4 or outside the device 10 as illustrated in FIG. 1. The comparator 220 determines if the target surface area is experiencing vasodilation. If the surface area is not experiencing vasodilation, the comparator transmits an on-signal for the negative pressure generator 140 to at least throttle the desired negative pressure in the negative pressure chamber 104 to vasodilate the target surface area.

### Negative Pressure

Applying a negative pressure condition to a portion of the body can lower the vasoconstriction temperature and/or increase vasodilation in the body portion. In practicing the exemplary methods, the negative pressure conditions may be provided using any convenient protocol. In many embodiments, the negative pressure conditions are provided by enclosing a body portion of the mammal in the negative pressure chamber 14, where the pressure is then reduced in the sealed enclosure thereby providing the desired negative pressure that includes a target heat exchange surface. In many examples of the present methods and systems, the portion that is sealed includes an arm or leg, or at least a portion thereof, e.g., a hand or foot. The nature of the enclosure will vary depending on the nature of the appendage to be enclosed, where representative enclosures include gloves, shoes/boots, or sleeves.

Negative pressure includes conditions where a pressure lower than ambient pressure under the particular conditions in which the method is applied, e.g., 1 ATM at sea level. The magnitude of the decrease in pressure from the ambient pressure under the negative pressure conditions in one example is at least about 20 mmHg, preferably at least 30 mmHg, and more preferably at least about 35 mmHg, where the magnitude of the decrease may be as great as 85 mmHg or greater, but preferably does not exceed about 60 mmHg, and more preferably does not exceed about 50 mmHg. When the method is performed at or about sea level, the pressure under the negative pressure conditions generally may range from about 740 to 675 mmHg, preferably from about 730 to 700 mmHg and more preferably from about 725 to 710 mmHg.

In practicing the exemplary methods, the negative pressure conditions during contact with the skin of a subject may be static/constant or variable and no matter what is turned on in response to the sensor signal. Thus, in certain examples, the negative pressure is maintained at a constant value during contact of the surface with the low temperature medium to obtain the desired vasodilation. In yet other examples, the negative pressure value is varied during contact, e.g., oscillated. Where the negative pressure is varied or oscillated, the magnitude of the pressure change during a given period may be varied in response to the sensor signal and may range from about 85 to 40 mmHg, and preferably from about 40 to 0 mmHg, with the periodicity of the oscillation ranging from about 0.25 sec to 10 min, and preferably from about 1 sec to 10 sec.

### Alternative Components

The device 10 may also have a systems controller 400 that provides and receives signals from the various system components to achieve controlled thermal energy transfer from at least a portion of the patient and/or control the vasodilation through the negative pressure applied to the target surface area. The systems controller 400 may include a unit having a suitably programmed microprocessor or the like, including algorithms or program logic for various heating, normothermic, and cooling protocols and schedules as desired. The algorithms may be carried out through software, hardware, firmware, or any combination thereof. The programming can be recorded on computer readable media, (e.g., any medium that can be read and accessed directly by a computer). Such media include, but are not limited to: magnetic storage media, such as floppy discs, hard disc storage medium, and magnetic tape; optical storage media such as CD-ROM; electrical storage media such as RAM, ROM, or an EPROM; and hybrids of these categories such as magnetic/optical storage media. Any such medium (or other medium) programmed (in full or in part) to operate according to the subject methodology also forms an aspect of the invention.

In some embodiments, the systems controller 400 is in communication with the vacuum generator 140 and the thermal exchange engine 180 which is capable of heating or cooling a heat exchange medium (not shown) in communication with the within the cavity 36. The heat exchange medium provided may communicate thermally with at least a portion of the mammal and with at least a portion of the conductor 32. In certain examples, the heat exchange medium is comprised of a fluid such as water, oil, and the like. In other examples the heat exchange medium may include gas or air. In further examples, the heat exchange medium may include solid-state heating or direct electrical heating. Additionally, the systems controller is in communication with a reservoir (not shown) for containing a supply of heat exchange medium.

Though the invention has been described in reference to several examples, optionally incorporating various features, the invention is not to be limited to that which is described or indicated as contemplated with respect to each embodiment or variation of the invention. It will be apparent to those skilled in the art that numerous modification and variations within the scope of the present invention are possible. Thus, the breadth of the present invention is to be limited only by the literal or equitable scope of the following claims—not the description provided herein.

We claim:

1. A device for introducing thermal energy into the core body of a mammal, the device comprising:
(a) a sensor for (i) measuring and/or detecting a requirement for vasodilation in the mammal and (ii) transmitting a sensor signal regarding the measurement and/or detection of the requirement for vasodilation in the mammal;
(b) an enclosure for enclosing a portion of the mammal, the enclosure has a seal that (i) leaks or (ii) does not leak, the seal retains a negative pressure within the enclosure;
(c) a negative pressure generator that initiates the negative pressure to a predetermined pressure within the enclosure (i) in response to the sensor signal that vasodilation is required and (ii) to control the vasodilation of at least a part of the portion of the mammal within the enclosure.

2. The device of claim 1 further comprising a thermal energy provider that provides thermal energy at a predetermined temperature to at least a part of the portion of the mammal within the enclosure.

3. The device of claim 2 wherein the thermal energy provider is independent from the sensor signal.

4. The device of claim 1 wherein the negative pressure to cause vasodilation is between −20 to −85 mm Hg.

5. The device of claim 1 wherein the negative pressure generator alters and/or maintains the negative pressure in the enclosure to obtain the desired vasodilation.
